(19) United States
(12) Patent Application Publication
(10) Pub. No.: US 2016/0310634 A1
(43) Pub. Date: Oct. 27, 2016

(54) A PROCESS FOR THE PREPARATION OF TITANIUM FOAM

(71) Applicant: COUNCIL OF SCIENCETIFIC & INDUSTRIAL RESEARCH, New Delhi (IN)

(72) Inventors: Gaurav Kumar GUPTA, Bhopal (IN); Mohit SHARMA, Bhopal (IN); Om Prakash MODI, Bhopal (IN); Braj Kishore PRASAD, Bhopal (IN)

(21) Appl. No.: 14/903,133

(22) PCT Filed: Jan. 6, 2015
(86) PCT No.: PCT/IN2015/050002

§ 371 Date: Jan. 6, 2016

(30) Foreign Application Priority Data
Jan. 6, 2014 (IN) 0033/DEL/2014

(51) Int. Cl. A61L 27/06; B22F 3/11; C22C 1/08; A61L 27/56

(52) U.S. Cl. CPC A61L 27/06; A61L 27/56; B22F 3/1125; C22C 1/08; A61L 2430/02; A61L 2400/08

(57) ABSTRACT

The disclosure relates to a process for the preparation of a titanium foam through a powder metallurgy route using Acrawax particles as a space holder material. An open cellular titanium foam is provided, having desirable porosity and good mechanical properties. The titanium foam is useful as a bone implant material.

### FIELD OF THE INVENTION

The present invention relates to a process for the preparation of titanium foam useful as bone transplant material through powder metallurgy route, using Acrawax particles as the space holder material. The Present invention provides open cellular titanium foam having desirable porosity and good mechanical properties.

### BACKGROUND OF THE INVENTION

During recent years, open cellular titanium foam are in greater demand due to their possible use as bone implants and various other engineering applications such as heat exchangers and catalyst substrates (D. C. Dunand, Adv. Eng. Mater. 6, 369, 2004). The foam have been developed employing various powder metallurgy (P/M) processing techniques. The basic technique for developing foam involves partial sintering of loosely compacted Ti powder. However, it becomes difficult to control the size and shape of the pores in this case (D. C. Dunand, Adv. Eng. Mater. 6, 369, 2004). In another approach, entrapped gas expansion technique has been used to fabricate titanium foam. However, the pore interconnectivity in this case adversely affected in general with interconnectivity obtained at high pore fractions only (N. G. D. Murray et al. Compos. Sci. Technol. 63, 2311, 2003).

U.S. Pat. No. 6,660,224 discloses the use of foaming agents like carbonates and hydrides to develop open cellular foam. In this approach, metallic powder was mixed with an organic solid binder and the foaming agent. The mixture is foamed at a temperature where the organic binder gets melted. The foamed material is then heated to eliminate the organic binder. This is followed by sintering at high temperatures to produce the open cellular foam. However, it becomes difficult to control the shape, size and interconnectivity of the pores in the matrix applying this approach.

Space holder technique is a widely used process for producing open cellular foam wherein the space holder material gets evaporated at a lower temperature leaving behind an interconnected porous structure. The space holder technique offers a good control over the pore content and morphology by varying the shape and size of the space holder material. Various space holders like NaCl, tapioca starch, magnesium, saccharose and carbamide particles have been utilized for synthesizing open cellular titanium foam (N. Jha et al. Mater. Des. 47, 810, 2013; A. Mansourighasri et al. J. Mater. Process. Technol. 212, 83, 2012; Z. Esen et al. Scr. Mater. 56, 341, 2007; J. Jakubowicz et al. J. Porous Mater. DOI 10.1007/s10934-013-9696-0, 2013; B. Jiang et al. Mater. Lett. 59, 3333, 2005). The space holder materials get removed from the matrix in such cases either by way of dissociation, burning off or getting dissolved in some solution. The first kind of space holders includes ammonium bicarbonate and carbamide particles which get dissociated into carbon dioxide and ammonia. In one of the earlier studies, titanium powder was mixed with 50 vol.% ammonium bicarbonate and cold compacted at 200 MPa. The synthesized titanium foam (58% dense) possessed 24.6 GPa Young's modulus and 215 MPa yield strength which were comparable with those of the mechanical properties of cortical bone. It has also been stated that the use of higher compaction pressure (up to 800 MPa) increases the inter-connectivity of the open cellular pores (Y. Torres et al. J. Mater Sci. 47, 6565, 2012). However, this process has disadvantages like dissociation of the space holder yielding gases which are not environment friendly. In order to resolve the issue, organic tapioca starch has been used as the space holder material. This space holder material burns off during processing, is chemically stable and does not react with the titanium matrix. The so processed foam contained porosity in the range of 64-79 vol % and Young's modulus 1.6-3.7 GPa (A. Mansourighasri et al. J. Mater. Process. Technol., 212, 83, 2012). The other class of space holders is readily soluble in water and leaves empty space after dissolution. In one of the processing approaches, the dissolution of the space holder material (NaCl) takes place after the partial densification of titanium. The process involves four different processing steps including the debinding of the inorganic lubricant, a two-stage sintering process at 800 and 1100° C. and removal of NaCl a hot water after sintering at 800° C. The developed foam contained 60-80% porosity with cell size ˜250 μm and Young's modulus 8-15 GPa. The mentioned characteristics of the developed foam render them suitable for applications such as bone scaffolds (N. Jha et al. Mater. Des. 47, 810, 2013). However, there is a chance in this process that some of titanium might react with NaCl especially at high temperatures leading to poor corrosion resistance and mechanical properties.

In the year 2009, a U.S. Pat. No. 2009/0096,121 disclosed the use of sodium chloride, polyethylene oxide (PEO), low density polyethylene (LDPE) thermoplastic material and reticulating (dicumyl peroxide) agent, all in powder form, for processing open cellular titanium, nickel and copper foam. The mixture of the powders was heated to crosslink the LDPE thermoplastic binder, removal of NaCl and PEO in warm water followed by two step sintering process at 420 and 1000° C. for 2 and 1 hr respectively in argon atmosphere. This method ensured lesser reactivity of NaCl since it was removed prior to the sintering process at high temperatures. The strength of the compact after the removal of NaCl was maintained with the cross-linking LDPE polymer. However, again the burning off the LDPE polymer releases harmful gases which are not environment friendly. In a further improvement of the above processes, saccharose particles were used as the space holder material which involved their removal using hot water (J. Jakubowicz et al. J. Porous Mater. DOI 10.1007/s10934-013-9696-0, 2013). However, saccharose was removed in this case just after the cold compaction of the powder mixture. In order to ensure enough green strength of the powder compacts after removal of sugar, the powder mixture was cold compacted at a high compaction pressure of 500 MPa. However, the use of higher compaction pressure led to the fracturing of the brittle saccharose crystals resulting into the formation of irregular macro-pores. Moreover, some amount of residue was also left behind thus adversely affecting the sinterability of titanium.

It may be noted that although space holder route is quite beneficial in the formation of the open cellular foam yet there are certain limitations such as (1) carbamide, NaCl are hygroscopic in nature making them unsuitable for easy handling in humid conditions. Moreover, their use as the space holder material will cause the oxidation of titanium during their removal at higher temperatures, (2) the space holder material such as carbamide, ammonium carbonate get removed forming polluting gases such as ammonia and carbon monoxide and (3) leachable type space holder materials such as sodium chloride and saccharose particles are difficult to remove completely if present isolated in the matrix. The remnant isolated particles might lead to poor mechanical properties. To keep the foam structure intact during complete removal of NaCl in hot water, a higher compaction pressure is desired. However, the compressibility of water leachable space holders is generally poor and they fracture upon using higher compaction pressure leading to irregular pores in the matrix.

In view of the above, it becomes important to use a space holder material which has certain advantages over the mentioned ones and can avoid/minimize associated problems. One of such materials could be acrawax. This material has been used over the past as a lubricant for easier cold compaction of stainless steel powder in China Pat. No. CN 101259530 A and aluminium alloy powders (G. B. Schaffer et al. Acta Mater. 54, 131, 2006). It has been demonstrated in earlier studies that acrawax gets removed completely during de-binding by evaporation and not by dissociation. Further, its compressibility is also good as compare to other space holder as it deforms plastically even at higher compaction pressures. Accordingly, open cellular foam of varied pore size and fraction can be produced using Acrawax of appropriate size and quantity as the space holder material.

### OBJECTS OF THE INVENTION

The main objective of the present invention is to provide a process for the preparation of titanium foam having desirable properties for cortical bone applications.

Another objective of the present invention is to generate interconnected network of pores with uniform spherical shape and size.

Yet another objective of the present invention is to use a space holder material which does not leave behind any residue after removal.

Further objective of the present invention is to use a space holder material which does not dissociate leading to gas formation.

Yet another objective of the present invention is to use a space holder material which is condensed for its reuse after getting evaporated.

Further objective of the present invention is to use a space holder material which sustains high compaction pressures and acts as a lubricant for easier die ejection.

### SUMMARY OF THE INVENTION

Accordingly, the present invention provides a process for the preparation of titanium foam using a compressible, lubricating and re-collectable type space holder (acrawax) material wherein the said process comprises of following steps:

a) Providing acrawax particle,

b) Providing titanium particle,

c) Providing iso-propanol, d) mixing acrawax particle obtained in step (a) with titanium particle obtained in step (b) for a period of 1-2 hours,

e) adding iso-propanol obtained in step (c) during mixture preparation of said step (d),

f) performing cold compaction on a mixture obtained in step (d) at 60-200 MPa minimum for 30 seconds,

g) pre-heating the mixture obtained in step (f) at 280-300 degree temperature for a period of 2-3 hours,

h) sintering the foam formed in step (g) at 1100-1200° C. for 1-2 hours to obtain titanium foam.

In an embodiment of the present invention the size of the acrawax particles is in the range of 200-1000 μm.

In an embodiment of the present invention the size of the titanium particles is in the range of 20-100 μm.

In still another embodiment to the present invention, the amount of the iso-propanol is in the range of 1-2 wt %.

Yet another embodiment to the present invention, the titanium foam has porosity content in the range of 40-70 Volume %.

In still another embodiment to the present invention, the titanium foam obtained is open cellular titanium foam.

Yet another embodiment to the present invention, titanium foam is used as a bone implant material.

### BRIEF DESCRIPTION OF THE FIGURES

FIG. 1 DSC/TGA thermograph of the used acrawax particles showing (1) softening of acrawax (2) melting (3) Evaporation (4) burning off.

FIG. 2 Micrographs of titanium and acrawax powder mixture cold compacted at (a) retained sphericity of acrawax at 200 MPa showing and (b) initiation of deformation in acrawax at 250 MPa [marked by arrows].

FIG. 3 Micrographs of the processed open cellular titanium foam with coarse pores showing (a) general distribution and (b) interconnectivity of pores in the matrix.

FIG. 4 Micrograph showing interconnected pores in the case of Ti foam with fine pores showing (a) general distribution and (b) interconnectivity of pores in the matrix.

FIG. 5 Compressive stress-strain diagram of the titanium foam with varying porosity (40, 50, 60 and 70 vol %) levels synthesized using acrawax addition of size 500-1000 micron.

FIG. 6 Young's modulus versus relative density of the titanium foam synthesized using acrawax particles in the size ranges of 200-500 and 500-1000 microns.

FIG. 7 X-ray diffraction pattern of sintered titanium foam (*=Titanium).

### DETAILED DESCRIPTION OF THE INVENTION

The present investigation deals with the synthesis of open cellular Ti foam using a space holder material which leaves no residue nor does it dissociate into harmful gases during its removal. Instead, it evaporates and therefore can be condensed back. This space holder material also acts as a lubricant during compaction and eases the synthesis of open cellular foam.

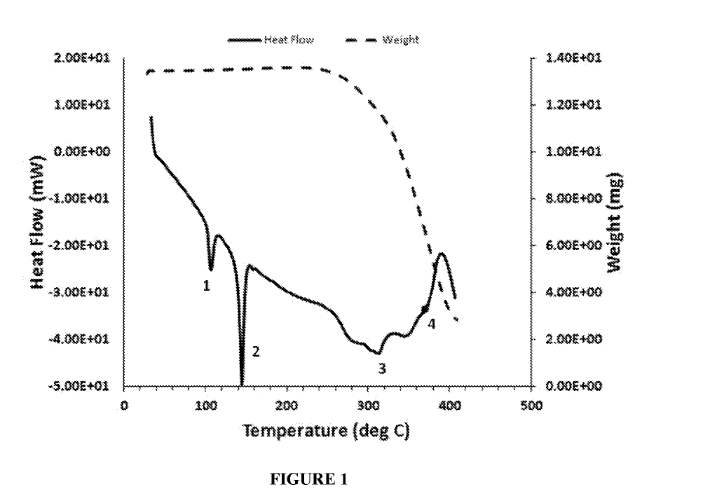

Processing details are as follows:
i. Preparation of the mixture of acrawax particles and titanium and by adding iso-propanol drop by drop during mixing to ensure the formation of a thin layer of titanium powder on the surface of acrawax particles, the mixing operation carried out for a period of 1-2 hrs Turbula mixer
ii. Cold compaction of the powder mix at 60-200 MPa pressure to shape it in the form of 10 mm diameter and 10 mm long cylindrical samples for compression tests and 50 mm length and 12 mm width for Young's modulus measurements
iii. Pre-heating the cold compacted samples at 280-320° C. for a time period of 2 hrs in air using a tubular furnace having gas inlet and exit; a provision was made to recollect the acrawax at the gas exit.
iv. Reweighing the samples to ensure the complete removal of wax through weight loss measurements prior to and after pre-heating as also confirmed by DSC/TGA analysis (FIG. 1).
v. Sintering the pre-heated samples at 1100° C. for one hr
vi. Open cellular titanium foam developed after the said processes (i) to (v)

### Examples

The following examples are given to illustrate the process of the present invention and should not be construed to limit the scope of the present invention.

### Example 1

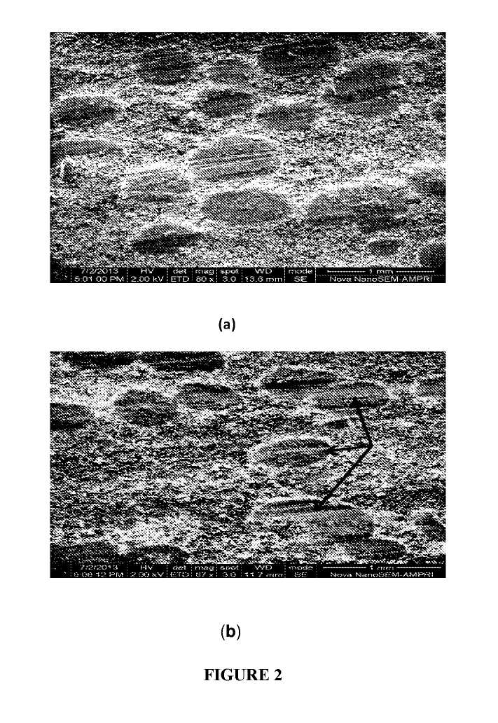

A powder mixture containing 50 vol % acrawax (special particles, size range: 500-1000 micron) and titanium (irregular shaped particles, size range: 15-40 micron) were cold compacted by varying the compaction pressure between 60-300 MPa. This study was performed to observe the compressibility and shape retention of acrawax. Below 60 MPa, the samples did not possess enough green strength and became fragile. On increasing the compaction pressure above 60 MPa, the cold compacted samples became easier to handle and no shape or size changes were observed in the acrawax particles (FIG. 2a). At applied pressures beyond 200 MPa, initiation of compression of the acrawax particles was observed (FIG. 2b). However, the use of a higher compaction pressure did not lead to any cracking or fracture in the acrawax particles but instead reduced their sphericity. Therefore, in order to retain the spherical shape of acrawax, it was important that the compaction pressure lies in the range of 60-200 MPa (FIG. 2a). Accordingly, the compaction pressure employed in this investigation for preparing the samples for characterization was varied in the range of 60-200 MPa.

### Example 2

A powder mixture containing 30-60 vol % titanium powder (particle size range 15-40 micron), 40-70 vol % acrawax (particle size range 500-1000 micron) and 1 wt % iso-propanol was prepared through conventionally mixing in a turbula mixer. The powder was cold compacted at 60-200 MPa. After the removal of acrawax at 300° C., the foam were sintered at 1100° C. for 1 hr.

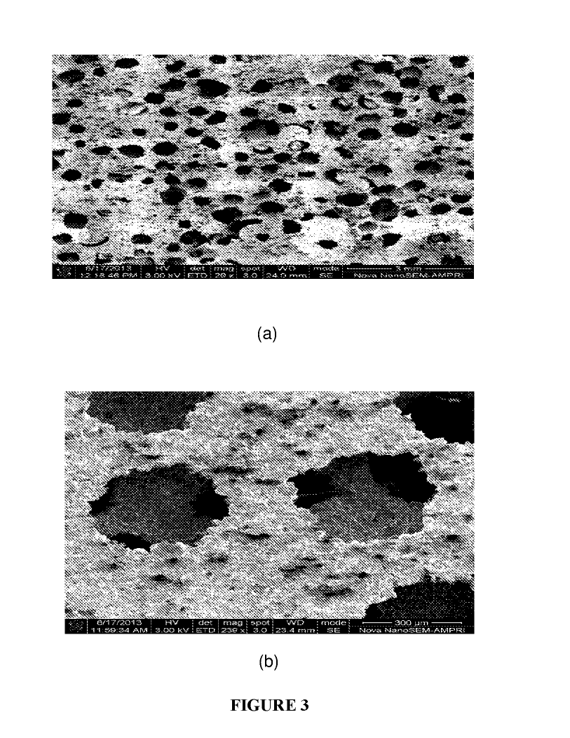

FIG. 3a shows the microstructure of the open cellular titanium foam formed after the sintering process. The interconnected porosity (FIG. 3b) symbolizes the formation of the open cellular network of the pores. The titanium foam possessed cell thickness of 200-300 micron and pore size range 300-600 micron.

### Example 3

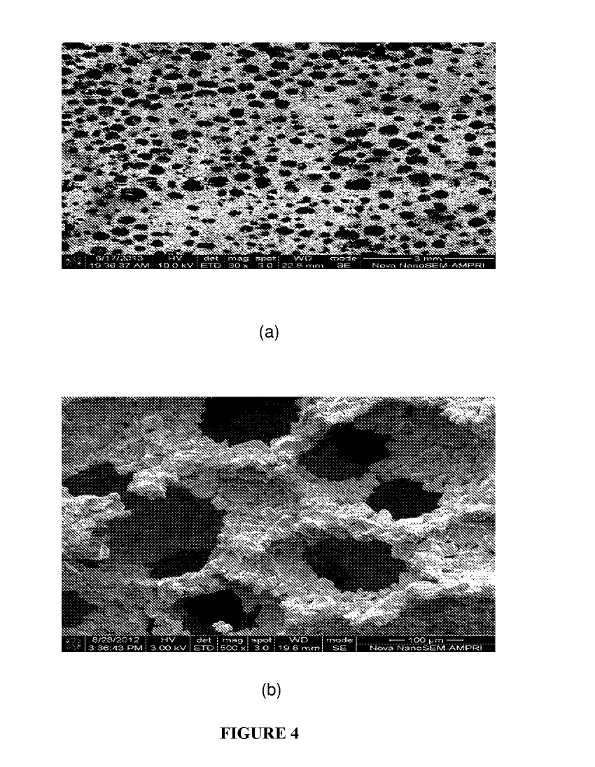

A powder composition containing 65 to 90 wt % of titanium powder (particle size range 15-40 micron) and 10 to 35 wt % of Acrawax, lonza India (particle size range 200-500 micron) and 1 wt % iso-propanol was prepared by conventionally mixing in a turbula mixer. The powder was cold compacted at 60-200 MPa. After the removal of acrawax at 300° C., the foam were sintered at 1100° C. for 1 hr. FIG. 4a shows the microstructure of the open cellular titanium foam formed after the sintering process. The interconnected porosity seen in FIG. 4b symbolizes the formation of the open cellular network of the pores. The titanium foam possessed cell thickness of 70-120 micron and the pore size ranged from 150 to 350 micron.

### Example 4

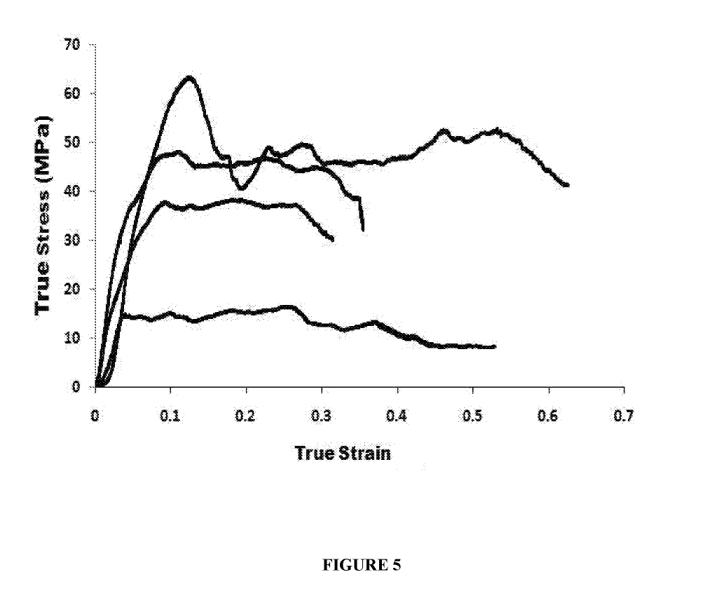

Titanium foam synthesized as per the procedure mentioned in Example 2 showed a porosity content of 40, 50, 60 and 70 vol % depending on the used volume fraction of the space holder (acrawax). The foam were subjected to compression testing in the quasi-static state at a strain rate of 10⁻³/sec. The yield strength of the foam sample decreased from 65 to 15 MPa with increasing pore fraction from 40 to 70 vol % (FIG. 5).

### Example 5

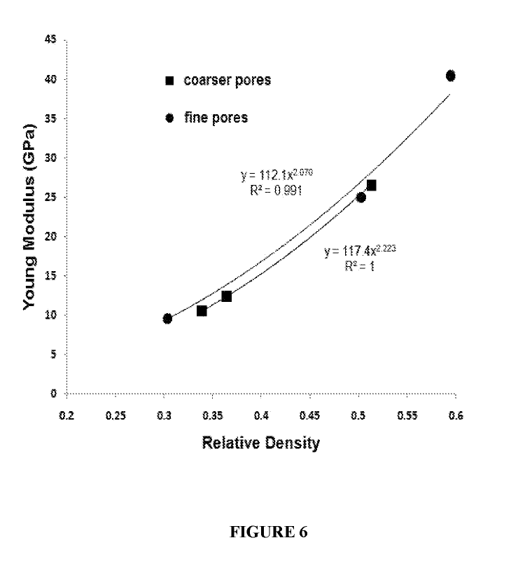

Titanium foam with coarser and finer pores (corresponding pore size ranges being pore 300-600 and 150-350 micron respectively) were synthesized with a porosity content of 40, 50, 60 and 70 vol % using the procedures shown in example 2 and 3 respectively. The Young's modulus of the foam with coarser pores increased from 10 to 26 GPa when the level of porosity decreased from 70-50% (FIG. 6). Similarly, the foam with finer pores attained higher Young's modulus with decreasing porosity. The observed range of the modulus was noted to be 10-42 GPa for a porosity range of 40-70% (FIG. 6).

### Example 6

The Young's modulus in Ti foam with both finer and coarser pores follows a power law correlation with the relative density with the coefficient of regression close to 1, thereby signifying uniform distribution of pores in the samples with good reproducibility of property. The pre-exponential coefficient (112-117 GPa) is almost matching with the Young's modulus of dense Ti (120 GPa). This indicates that there is no appreciable defect present in the cell wall due to complete sintering of titanium. Further, only fine pores existed in marginal quantity in the cell wall as also evident from the microstructural features of the samples (FIGS. 3b & 4b). It may be noted that defect-free structure is possible only when the space holder gets removed completely and there is good adherence between the Ti particles in the as compacted condition.

### Example 7

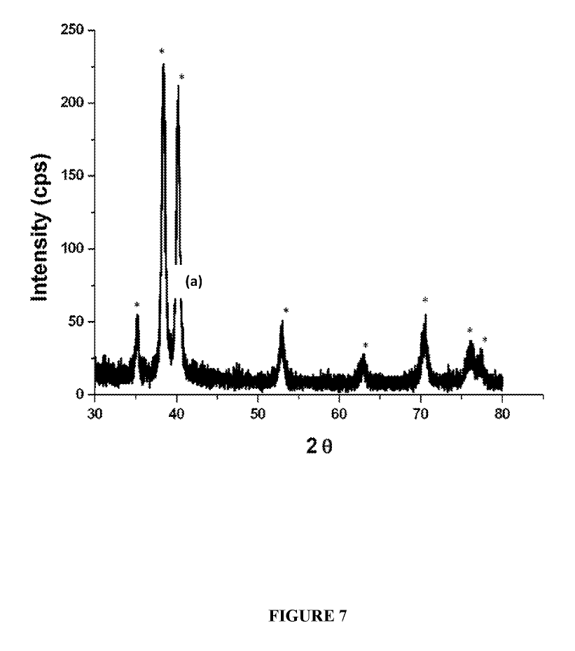

The X-ray diffractogram (FIG. 7) of the sintered foam shows only Ti peaks thus suggesting no oxidation of Titanium during the process of synthesizing the foam. It is also confirmed from the observed value of the pre-exponential coefficient that came to be ˜1 (FIG. 6) that the cell walls contain only Ti for all practical purposes and not oxides of Titanium.

Advantages of the present invention are:
(i) The used process produces open cellular titanium foam having pore size and mechanical properties (Young's modulus and yield strength) having potential for use as bone implants.
(ii) The used space holder material (a) does not leave behind any residue during its removal, (b) does not dissociate to form green house gases, (c) is also collectable after getting evaporated, (d) is compressible under compaction pressures, (e) acts as a lubricant for easier die ejection and (f) is also available in larger size ranges in spherical shape thus making it possible to synthesize open cellular foam with various cell size ranges.
(iii) The Young's modulus correlation with relative density was truly following the power law having coefficient of regression ˜1 in the Ti foam with both finer and coarser pores. Further, the pre exponential coefficient of the synthesized foam samples (112-117 GPa) closely matched with that of the of dense Ti (120 GPa).

1. A process for the preparation of a titanium foam comprising of:
a) obtaining an acrawax particle,
b) obtaining a titanium particle,
c) obtaining iso-propanol,
d) mixing the acrawax particle obtained in step (a) with the titanium particle obtained in step (b) for a period of 1-2 hours,
e) adding the iso-propanol obtained in step (c) during the mixing of said step (d),
f) performing cold compaction on a mixture obtained in step (d) at 60-200 MPa minimum for 30 seconds,
g) pre-heating the mixture obtained in step (f) at 280-300° C. degree temperature for a period of 2-3 hours, wherein a foam is formed,
h) sintering the foam formed in step (g) at 1100-1200° C. for 1-2 hours to obtain the titanium foam.

2. The process as claimed in claim 1, where in the size of the acrawax particle is in the range of 200-1000 μm.

3. The process as claimed in claim 1, wherein the size of the titanium particle is in the range of 20-100 μm.

4. The process as claimed in claim 1, where in the amount of the iso-propanol is in the range of 1-2 wt %.

5. The process as claimed in claim 1, where in the titanium foam has a porosity content in the range of 40-70 Volume %.

6. The process as claimed in claim 1, wherein the titanium foam obtained is an open cellular titanium foam.

7. A process of forming a bone implant comprising:
performing the process according to claim 1, and
using the titanium foam as a bone implant material.

8. A titanium foam prepared by using the process as claimed in claim 1.

9. A titanium foam having an open cellular network of pores and a porosity content in the range of 40-70% by volume.

10. The titanium foam according to claim 9, wherein the titanium foam comprises cell thicknesses of 200-300 μm and pore sizes in the range of 300-600 μm.

11. The titanium foam according to claim 9, wherein the titanium foam comprises cell thicknesses of 70-120 μm and pore sizes in the range of 150-350 μm.

12. The titanium foam according to claim 9, wherein titanium foam has a Young's modulus of from 10-42 GPa.
